(19) United States
(12) Patent Application Publication
(10) Pub. No.: US 2004/0220506 A1
(43) Pub. Date: Nov. 4, 2004

(54) Designer terri stix the new and improved way of cleaning

(76) Inventors: Depresha Sharay Gordon, Pittsburg, CA (US); Gail Sue Gordon, Pittsburg, CA (US)

Correspondence Address: Depresha Sharay Gordon, 614 Chester Drive, Pittsburg, CA, 94565, US

(21) Appl. No.: 10/334,077

(22) Filed: Apr. 30, 2003

(51) Int. Cl. A61M 35/00

(52) U.S. Cl. 604/1

(57) ABSTRACT

A Designer Terri Stix is a new and improved way of cleaning. The function of this product is to sanitize the ear canal more safely preventing the terry cloth fabric from unraveling and becoming caught inside the auditory canal. This also can be utilized for tedious areas as well. A Designer Terri Stix is described whose stick is an elongate stem either of plastic, wood, or paper materials. The terry cloth fabric is key to the uniqueness of this product.

### BACKGROUND OF THE INVENTION

1. Field of the Invention

The invention relates to cotton swabs useful in cleaning the ear or applying cosmetics.

2. The Related Art

Generally Swabs have absorbent material such as cotton. An adhesive may be used to more firmly hold the absorbent material in place on the Swab. However, the patent suggest that injuries may be prevented by positioning a flat disc at each end of the Swab beneath the cotton material, but the flat disc increases the rigidity of the cotton covered end tips rendering the ends harder.

Performance discrepancies have long been related with Swabs. U.S. Pat. No. 5,127,899 (Schmerse Jr.) who has raised the question of ear drum damage when Swabs are improperly applied to clean the ear.

It is evident that further improvements are necessary in products for the ear technology as well as for a cleaning tool. The improvements should focus on making sure that the product has larger ends and it is tightly secured where it won't unravel and become caught inside the ear canal.

Designer Terri Stix includes a supportive elongate stem. This can be manufactured of plastic, wood, or paper materials having terry cloth fabric on opposite ends of one another wrapped outwardly. Although, utilizing an adhesive generates my innovation, the object is for the terry cloth cotton not to unravel and become caught inside the ear while utilizing this product. For instance, there have been several cases involving cotton swabs consumers having to see a physician for the removal of cotton inside the ear canal. This is an assured way preventing this from occurring. Designer Terri Stix is broadly utilized for example for sanitary purposes relating to tedious areas and various applications. New uses are: the rims of a tire, heater vents, to clean office equipment, window blinds, window panels, and baby creases. This also can be used in veterinarian facilities.

Traditionally, this methodical way of sanitizing the ear canal is conducted with a terry cloth towel while showering, opposed to having to shower to clean your ears thoroughly. The revolutionary upgrade of this new and improved way of cleaning will now become available as a portable utility product. This new and improved way of cleaning is also larger tips and more secure safe product because the terry cloth fabric will not unravel and become caught inside of the ear canal and cause eardrum damage. This is why it is evident that further improvements are once again essential in ear technology.

### SUMMARY OF THE INVENTION

A further aspect of the invention is that of a process for preparing Designer Terri Stix.

Steps of this process include:

An elongate stem that can be manufactured of plastic, wood, or paper materials.

Terry cloth fabric is placed at opposite ends of the stem. This process transpires by the terry fabric being wrapped outwardly and adhering with an adhesive until it begins to take its form.

### BRIEF DESCRIPTION OF THE DRAWING

The drawings being view perspective consist of:

FIG. 1. Which is a plan perspective view of an elongate stem that can be manufactured of plastic, wood, or paper materials according to the present invention.

FIG. 2. Which is a plan perspective view of terry cloth fabric rendered for an adult size.

FIG. 3. Which is a plan perspective view of the Designer Terri Stix with the first and second ends opposite one another with terry cloth fabric wrapped outwardly adhering with an adhesive according to the present invention.

### DETAILED DESCRIPTION

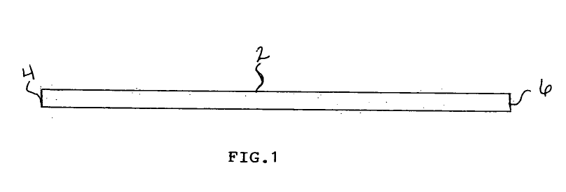

FIG. 1. Illustrates an elongate stem 2 with first and second ends 4,6 at opposite square ends from one another.

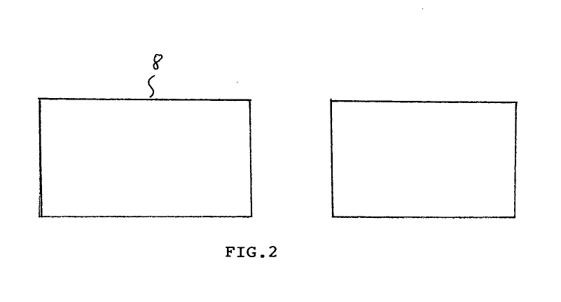

FIG. 2. Illustrates the terry cloth fabric 8

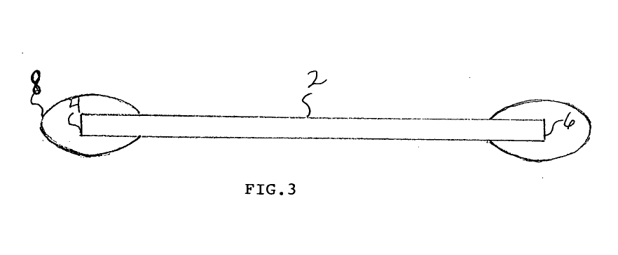

FIG. 3. Illustrates the Designer Terri Stix complete structure. Terry cloth fabric covering 8 which is wrapped outwardly on opposite ends of the elongate stem.

What is claimed as my invention:

1. Designer Terri Stix is comprising:
An elongate stem manufactured plastic, of wood, or paper materials with terry cloth fabric on first and second ends opposite one another wrapped outwardly this procedure is prepared by utilizing an adhesive to adhere to create a tight secure custom fit to prevent the terry cloth fabric from unraveling and becoming caught inside of the ear canal the terry cloth fabric is also larger and more durable this method sanitizes the ear canal more thoroughly and effectively it provides versatility for the product many uses such as having access tedious areas the terry cloth fabric is key to the uniqueness of this product the revolutionary upgrade of this new and improved way of cleaning will now become a portable product.

2. A Designer Terri Stix according to claim 1 wherein the fabric covering is terry cloth fabric.

3. A Designer Terri Stix according to claim 1 will not unravel and become caught inside of the ear canal.

4. A Designer Terri Stix according to claim 1 wherein is larger terry cloth tips and more durable.

5. A Designer Terri Stix according to claim 1 wherein the terry cloth fabric sanitizes more thoroughly and effectively.

6. A Designer Terri Stix according to claim 1 wherein provides versatility for the product many uses having access to tedious areas.

7. A Designer Terri stix according to claim 1 wherein is a revolutionary upgrading of this new and improved way of cleaning into a portable utility product.
A further aspect of the invention is that of a process for preparing Designer Terri Stix Steps of the process include:
(A) An elongate stem that can be manufactued of plastic, wood, or paper materials.
(B) Terri cloth fabric is placed at opposite ends of the stem. This process transpires by the terry cloth fabric being wrapped outwardly and adhering with adhesive until it begins to form.
